(19) United States
(12) Patent Application Publication
(10) Pub. No.: US 2003/0077296 A1
(43) Pub. Date: Apr. 24, 2003

(54) SENSE ENHANCING TOPICAL GEL

(76) Inventors: Michael G. Denton, Dallas, TX (US); Michael G. Denton JR., Dallas, TX (US); Gerry R. Hurst, Dallas, TX (US)

Correspondence Address: Jack A. Kanz, 502 So. Cottonwood Drive, Richardson, TX, 75080, US

(21) Appl. No.: 10/000,866

(22) Filed: Oct. 24, 2001

(51) Int. Cl. A61K 35/78; A61K 31/198; A61K 9/00

(52) U.S. Cl. 424/400; 424/728; 514/565; 424/744

(57) ABSTRACT

A composition for topical application to female genitalia to stimulate and enhance sensitivity during coitus is formed in a gel which includes propylene glycol, aloe barbadensis leaf juice, hydroxyethylcellulose, L'Arginine, menthol, methylparaben, Lepidium meyenii root extract and/or Anemopegma mirandum extract.

Various compositions in the form of creams and the like have been developed for topical application to female genitalia to enhance sexual stimulation and sensitivity. Typically, such compositions comprise an emulsion or suspension of an L'Arginine, an amino acid, in a sterile lubricant such as propylene glycol. Such compositions are only minimally effective, however, because they are difficult to apply properly, do not promote direct absorption of L'Arginine, and are not adapted to condition the epithelial tissue of the clitoris and vaginal area to react to the active ingredients.

The present invention obviates the shortcomings of prior art compositions. In accordance with the invention, sense enhancing compositions are produced in the form of gels which are readily and easily applied topically to female genitalia, primarily the clitoris, prior to coition. The gel is comprised of a humectant solvent such as propylene glycol and a binder such as hydroxyethylcellulose and contains a preservative such as is methylparaben, an analgesic stimulant such as menthol, a skin-conditioning agent such as aloe barbadensis leaf juice, and L'Arginine. In the preferred embodiment the composition of the invention also includes aphrodisiacs such as ginseng root extract and catuaba extract.

The composition of the invention is preferably formed in a batch process in which appropriate amounts of propylene glycol and hydroxyethylcellulose are combined to form a gel. Since these components are not soluble in each other the batch must be agitated and maintained at about 90° C. for about eight (8) hours to form a satisfactory gel. The required portions of water, aloe barbadensis leaf juice, menthol, preservative and aphrodisiac extracts are mixed to form a solution which is added to the gel after the temperature of the gel has been reduced to less than 40° C. L'Arginine (and alanine, if used) is added to the composition only after the temperature of the batch is reduced to less than 30° C. to avoid decomposition of the amino acids. The final product is a stable creamy gel which is safe for human ingestion and has an indefinite shelf life.

While L'Arginine is known to increase sexual sensitivity when applied to the clitoris, effective use of L'Arginine as a sexual stimulant has been inhibited by lack of an appropriate system for applying the stimulant in a way which induces absorption and thereby enhancement of sensitivity. The composition of the present invention employs a skin-conditioning agent derived from aloe. The skin-conditioner appears to soften or sensitize the epithelial tissue of the clitoris to enhance the sensed effect of L'Arginine. The effect is even further magnified by small amounts of aphrodisiacs such as ginseng root extract and/or catuaba extract. An analgesic stimulant such as menthol, when included in the composition, not only stimulates immediate sensitivity in the female, it also induces pleasurable sensitivity in the penis of the male partner during coitus.

A composition which enhances and stimulates coital sensitivity may be formed in accordance with the invention using a humectant solvent such as propylene glycol, water, a skin-conditioning agent such as aloe barbadensis leaf juice, a binder such as hydroxyethylcellulose, an analgesic stimulant such as menthol, and a preservative such as methylparaben. Effectiveness of the composition can be increased by adding aphrodisiacs such as ginseng root extract and/or catuaba root extract. Alanine (in concentrations of from approximately 0.01% to approximately 0.9% by weight) may be substituted for or used in conjunction with L'Arginine to produce similar effects.

Various combinations of the composition have been compounded and tested.

The most satisfactory results have been attained with gels of the following composition:

92.13% by weight propylene glycol

4.00% by weight purified water

1.00% by weight aloe barbadensis leafjuice

1.00% by weight hydroxyethylcellulose

1.00% by weight L'Arginine

0.60% by weight menthol

0.25% by weight methylparaben

0.01% by weight Lepidium meyenii root extract

0.01% by weight Anemopegma mirandum extract

However, variations in the preferred composition can also produce satisfactory results. For example, acceptable results can be obtained if the composition of the gel is within the following ranges:

| | |
|---|---|
| propylene glycol | approximately 90% to approximately 94% |
| | by weight |
| water | approximately 2.5% to approximately |
| | 4.5% by weight |
| aloe barbadensis leaf juice | approximately 0.75% to approximately |
| | 1.25% by weight |
| hydroxyethylcellulose | approximately 0.75% to approximately |
| | 1.25% by weight |
| L'Arginine | approximately 0.25% to approximately |
| | 1.25% by weight |
| menthol | approximately 0.40% to approximately |
| | 0.80% by weight |
| methylparaben | approximately 0.15% to approximately |
| | 0.45% by weight |
| Lepidium meyenii root extract | 0.00% to approximately 0.02% by weight |
| Anemopegma mirandum | approximately 0.00% to approximately |
| extract | 0.02% by weight |
| Alanine | approximately 0.00% to approximately |
| | 0.90% by weight. |

All possible combinations of the listed ingredients in all possible concentrations have not been tested. It is expected, however, that compositions wherein the composition of one or more ingredients is greater or less than the precise range listed may also prove satisfactory and thus such compositions are considered to be within the scope of the invention disclosed.

The gel of the invention is stable and easily packaged in consumer-sized containers which have an indefinite shelf life. Best results are obtained by direct application of the gel to the underside of the clitoris in the vaginal area by rubbing or gently massaging immediately prior to coital activity. As the active ingredients contact the vaginal area, immediate stimulation is achieved and sensitivity enhancement continues throughout the coital act.

It will be appreciated form the foregoing that the principles of the invention may be used in various forms to provide the benefits and advantages described. However, various changes in composition, for example, may produce variations in achieved and/or desired results. It will be understood, therefore, that even though numerous characteristics and advantages of the invention have been set forth in the foregoing description together with details of the composition, preparation and use of the invention, this disclosure is for illustrative purposes only. Various changes and modifications may be made, especially in matters of component concentration and composition, without departing from the spirit and scope of the invention as defined by the appended claims.

What is claimed:

1. A gel for topical application to female genitalia to enhance and stimulate coital sensitivity comprising:
(a) a humectant solvent;
(b) water;
(c) a skin-conditioning agent derived from aloe;
(d) a binder;
(e) L'Arginine;
(f) an analgesic stimulant; and
(g) a preservative.

2. A gel as defined in claim 1 further comprising ginseng root extract and/or catuaba extract.

3. A gel as defined in claim 1 further comprising alanine.

4. A gel as defined in claim 2 which:
said humectant solvent is propylene glycol;
said skin-conditioning agent is aloe barbadensis leaf juice;
said binder is hydroxyethylcellulose;
said analgesic stimulant is menthol;
said preservative is methylparaben;
said ginseng root extract is Lepidium meyenii root extract; and
said catuaba extract is Anemopegma mirandum extract.

5. A gel as defined in claim 4 wherein:
said propylene glycol comprises approximately 90% to approximately 94% of said gel by weight;
said water comprises approximately 2.5% to approximately 4.5% of said gel by weight;
said aloe barbadensis leaf juice comprises approximately 0.75% to approximately 1.25% of said gel by weight;
said hydroxyethylcellulose comprises approximately 0.75% to approximately 1.25% of said gel by weight;
said L'Arginine comprises approximately 0.25% to approximately 1.25% of said gel by weight;
said menthol comprises approximately 0.40% to approximately 0.80% of said gel by weight; and
said methylparaben comprises approximately 0.15% to approximately 0.45% of said gel by weight.

6. A gel as defined in claim 5 further comprising approximately 0.01% to approximately 0.90% alanine.

7. A gel for topical application to female genitalia to enhance and stimulate coital sensitivity comprising:
(a) approximately 92.13% by weight propylene glycol;
(b) approximately 4.00% by weight purified water;
(c) approximately 1.00% by weight aloe barbadensis leaf juice;
(d) approximately 1.00% by weight hydroxyethylcellulose;
(e) approximately 1.00% by weight L'Arginine;
(f) approximately 0.60% by weight menthol;
(g) approximately 0.25% by weight methylparaben;
(h) approximately 0.01% by weight Lepidium meyenii root extract;
(i) approximately 0.01% by weight Anemopegma mirandum extract.
